(19) United States
(12) Patent Application Publication
(10) Pub. No.: US 2026/0273132 A1
(43) Pub. Date: Sep. 17, 2026

(54) IMPLANT

(71) Applicant: JAGE RESEARCH & INNOVATIONS (PTY) LTD, PRETORIA (ZA)

(72) Inventor: Gert Jacobus Jordaan, Venterskroon (ZA)

(21) Appl. No.: 19/469,646

(22) PCT Filed: Mar. 28, 2024
(86) PCT No.: PCT/ZA2024/050011

§ 371 Date: Sep. 26, 2025

(30) Foreign Application Priority Data
Mar. 28, 2023 (ZA) 2023/03885
Jan. 25, 2024 (ZA) 2024/00839

(51) Int. Cl. A61L 27/24; A61F 2/44; A61L 27/36; A61L 27/54

(52) U.S. Cl. CPC A61L 27/24; A61F 2/44; A61L 27/3604; A61L 27/54

(57) ABSTRACT

This invention relates to an implant (10), including a support arrangement (12) manufactured from a form of a porous material, for receiving and supporting stem cells (14) in a supported condition, for receiving a medium in the form of a neural basal and neural induction medium mixture (20) and the support arrangement (12) being shaped and sized to conform to and be received complementally by an implant zone (16). The implant zone (16) being a space defined by an excised portion of a subject's spinal cord (18). The neural basal and neural induction medium mixture (20) provides nutrients for the development of the stem cells (14) and the growth factors, in form of neurotrophin 3, neurotrophin 4 and brain-derived neurotrophic factor (BDNF) encourage and control development of the stem cells (14), the growth factors typically being constituents of the medium mixture (20).

### TECHNICAL FIELD

This invention relates to an implant. In particular, this invention relates to an implant for use in regenerative medical treatments.

### BACKGROUND

Tissue injury in mammals can lead to temporary, impaired or permanent loss of tissue architecture and/or function. Natural repair of injured tissue involves two processes, i.e., regeneration and/or replacement, depending on the type of tissue. Regeneration relates to new growth, which in turn, restores tissue architecture and function in at least a portion of the injured or damaged tissue. Replacement relates to the building of connective tissue in a region of the injury which results in scarring and loss of tissue architecture and function. Examples of tissue that are capable of regeneration are, hematopoietic tissue, epithelia, liver, bone and muscle. Tissues such as the heart muscle, pancreas and spinal cord respond to injury by the formation of scars.

Despite muscle and bone being listed as regenerative tissue, regeneration may be impeded by the presence of large gaps caused by severe injury. Also, degeneration of tissue due to age or disease can impede regeneration, irrespective of the type of injury or tissue.

The nervous system comprises the central nervous system and the peripheral nervous system. Injury to the central nervous system often occurs in an instant and typically does not regenerate. However, due to neuroplasticity, the neural system can reorganise to restore at least partial function. Surgical intervention can reduce or prevent secondary damage by relieving pressure on the spinal cord or the brain. However, the damage caused by the primary injury can lead to long-lasting effects such as partial or complete paraphysis.

Injury to the peripheral nervous system can occur in an instant or over time. The chance of regeneration in the peripheral nervous system is greater and may occur over a considerable period of time. Surgical options for peripheral nerve damage include, but is not limited to, removal of scar tissue, direct nerve repair and grafting.

Regenerative medicine is a field of medicine that focuses on replacing, engineering, or regenerating cells, tissues or organs. Stem-cell therapy is a key aspect of regenerative medicine and offers a promising option for the engineering of new tissue or enhancing endogenous repair by regeneration. Stem-cell therapy typically involves harvesting stem cells from a subject's own body (autologous) or from a donor (allogeneic). Once harvested, the stem cells are processed and prepared for injection or implantation into the subject's body at the site of injury or disease. Like with any emerging technology, there are many challenges to overcome to achieve effective and efficient stem-cell therapy.

These challenges include the limited ability of stem cells to survive and differentiate effectively post-implantation. This may be due to a lack of endogenous stimulus to cause effective differentiation and/or a lack of nutrient supply for development and maturation. Furthermore, handling or difficulties in handling the stem cells may cause death of the cells during implantation. Even further, without proper support and guidance, the stem cells may fail to integrate into the surrounding tissue or differentiate into the desired cell types. This is especially the case in regions of the body where the flow of bodily fluid can dislocate implanted stem cells.

In light of these challenges, there is a clear need for technology that allows for, ease of handling of stem cells during implantation, support for stem cells during and after implantation, sufficient nutrient supply for development and maturation, stimulus for differentiation or continued differentiation of stem cells and support.

The present invention addresses at least some of the needs mentioned above.

### SUMMARY OF THE INVENTION

According to a first aspect of the invention, there is provided an implant including:
a support arrangement for,
receiving and supporting stem cells in a supporting condition;
receiving a medium which includes nutrients; and
receiving a growth factor for encouraging and controlling development of the stem cells,
wherein the support arrangement is capable of being sized and shaped to conform to and be received complementally by an implant zone in a subject.

For the purposes of this specification, resorbable is to be understood as the ability of a substance to be degraded and absorbed by a body. In addition, bioabsorbable is to be understood as being capable of absorption into living tissue.

The support arrangement may be manufactured or synthesised using biological, biochemical, biocompatible, hemostatic, resorbable and/or bioabsorbable material. The material may be porous and/or fibrous. The support arrangement may have an internal lattice-type structure. The support arrangement may be constructed by 3D printing or additive manufacturing. The support arrangement may be manufactured or synthesised from hygroscopic and/or absorbent material. The support arrangement may define receiving zones, preferably in the form of interstices or pores, in which the stem cells may be received and supported in the supported condition. The receiving zones may have a size in the range of 150 to 1000 μm in width. Upon hydration and/or absorption, the receiving zones may have a size in the range of 150 to 500 μm in width. A reduction in size of the receiving zones, upon hydration and/or absorption, may facilitate support of the stem cells in the supported condition. A hole may be defined in the support arrangement for allowing insertion of the stem cells into an inner region thereof. It is to be appreciated that the hole serves to encourage filling, saturation and/or distribution of the stem cells substantially throughout the support arrangement. The hole may extend substantially co-axially the support arrangement. The hole may be formed by the use of a thin elongate member such as a needle. A plurality of holes may be defined in the support arrangement for further encouraging filling, saturation and/or distribution of the stem cells throughout the support arrangement. Each of the plurality of holes may extend at varying depths within the support arrangement, preferably two thirds the length of the support arrangement. The support arrangement may have a generally cylindrical shape. The support arrangement may have a length in the range of 1 to 4 cm, preferably being 2 cm. The support arrangement may have a diameter in the range of 0.8 to 1.2 cm, preferably being 1 cm.

The support arrangement may be synthesised or manufactured from a natural polymer. The natural polymer may be in the form of any one or more of the group including, proteoglycan, gelatin-based substrate, graphene, oxidised cellulose, dextrose, hyaluronic acid, starch, graphene oxide, fibrin, an alginate-based substrate, chitosan, collagen and any combination thereof. The natural polymer may be in the form of collagen, preferably being in the form of resorbable collagen. In particular, the resorbable collagen may include purified crosslinked type 1 collagen. The collagen may be derived from bovine Achilles tendon. The collagen may be resorbed within 20 to 40 days post-implantation, preferably being within 30 days. The support arrangement may be in the form of an RCP Resorbable Collagen PLUG®.

The support arrangement may be synthesised or manufactured from of a synthetic polymer. The synthetic polymer may be in the form of any one or more of the group including, polystyrene, poly-l-lactic acid, polyester, polyethylene glycol, polycyanoacrylate, polyurethane, polystat polyglycolic acid and poly-dl-lactic-co-glycolic acid.

The support arrangement may be synthesised or manufactured from a ceramic material. The ceramic material may be in the form of any one or more of the group including, hydroxyapatite and tricalcium phosphate.

The support arrangement may be synthesised or manufactured from a combination of any one or more of a natural polymer, synthetic polymer and/or ceramic material.

The support arrangement may be sized and shaped by a process of moulding and/or cutting. A plurality of support arrangements may be provided for facilitating conformation thereof to implant zones of various sizes and/or shapes. The plurality of support arrangements may be arranged in abutment to form a substantially continuous structure, wherein the structure allows for the implant to conform to and be received complementally by the implant zone.

The implant zone may be a site of injury, damage or deterioration of tissue in the subject's body. The implant zone may be a space defined by any one of the group including a damaged, deteriorated, severed, torn and excised tissue in the subject's body. More specifically, the implant zone may be a space defined by the excision of a portion of the subject's spinal cord. Alternatively, the implant zone may be a space defined by a severed or excised portion of the subject's peripheral nerve. The subject may be a mammal.

For the purposes of this specification, development of the stem cell is to be understood as including any one or more of, differentiation, continued differentiation, proliferation, migration and multicellular morphogenesis. In addition, a determined stem cell is to be understood as a stem cell which is committed to form a specific tissue-type or cell-type and a partially differentiated stem cell is to be understood as a stem cell exhibiting early morphologically distinct characteristics of a specific tissue-type or cell-type or a cell expressing molecular markers indicative of becoming a specific tissue-type or cell-type.

The stem cells may be in the form of any one of the group including totipotent, pluripotent, multipotent and unipotent stem cells. Preferably, the stem cells may be multipotent stem cells. The stem cells may be at any stage during its differentiation toward being a specific cell-type of interest. The stage of differentiation may be any one or more of the group including, undifferentiated, determined, partially differentiated and fully differentiated stem cells. A mixture of stem cells at various stages of differentiation may be provided. The mixture may include any one or more of undifferentiated, determined, partially differentiated and fully differentiated stem cells. Preferably, the mixture may include partially differentiated and fully differentiated stem cells. It is to be appreciated that inclusion of the fully differentiated stem cells in the mixture may ensure that a sufficient quantity of a specific cell-type or tissue-type of interest is present in the implant.

The partially differentiated stem cell may exhibit early morphologically distinct characteristics of neural cells. The characteristic may be the formation of an axon hillock. The partially differentiated stem cell may express one or more molecular markers specific to neural cells. The molecular markers may be any one or more of the group including, NeuN, Tuji, Synapsin, PAX6, SOX1, SOX2, bActin, NESTIN, MAP2 and/or GFAP.

The fully differentiated stem cells may be in the form of oligodendrocytes or Schwann cells. The oligodendrocytes may be in the form of oligodendrocytes progenitor cells (OPCs) and/or premyelinating oligodendrocytes. It is to be appreciated that the fully differentiated oligodendrocytes or Schwann cells may promote myelination of one or more other stem cells, either during development and/or when fully developed. Further, it is to be appreciated that the fully differentiated oligodendrocytes may promote myelination or remyelination of endogenous neurons in a central nervous system of the subject, post-implantation, whereas the fully differentiated Schwann cells may promote myelination or remyelination of endogenous neurons in a peripheral nervous system of the subject, post-implantation. The endogenous neurons may be an impaired or damaged neuron in a region of the implant zone. The oligodendrocytes or more specifically, OPCs, may express any one or more molecular markers of the group including, SOX 10, NKX 2.2, OLIG1 and OLIG2. The oligodendrocytes or more specially, premyelinating oligodendrocytes, may express any one or more molecular markers of the group including, OLIG1, OLIG2, SOX10, NKX2.2, ZFP191, ZFP488, ZFP536, SOX17 and NKX 6.2.

The stem cells may have a concentration in a range of 150 000 to 1 500 000 per 1 cm³ of material. Surplus stem cells may be provided for allowing differentiation of the stem cells into the cell-type of interest or any other cell-type which may be formed as a consequence of cell-to-cell signalling or endogenous signalling post-implantation. In surplus, the stem cells may have a concentration in the range of 2 000 000 to 3 000 000 per 1 cm³ of material. The fully differentiated stems cell may comprise between 2 to 4% of a total number of stem cells, preferably being in the region of 3%. The stem cells may be obtained during any passage, preferably being any of passages 3 to 9. More specifically the fully differentiated stem cells may be obtained during passage 5 and/or 6. The stem cell may be at any phase in its cell cycle.

The stem cells may be in the form of any one or more of the group including embryonic stem cells (ESCs), mesenchymal stem cells (MSCs), hematopoietic stem cells (HSCs), neural stem cells (NSCs), induced pluripotent stem cells (iPSCs), dental pulp stem cells (DPSCs), olfactory ensheathing cells (OECs), cancer stem cells (CSCs) and/or human foetal brain neural stem cells (hNSCs). MSCs may include bone marrow mesenchymal stem cells (BM-MSCs), human umbilical cord-mesenchymal stem cells (HUC-MSCs), and adipose-derived mesenchymal stem cells (AD-MSCs). NSCs may include neural progenitors, neutral crest cells and/or Schwann cell precursors. Preferably, the stem cells may be in the form of MSCs and more preferably, AD-MSCs. The MSCs or the AD-MSCs may be identifiable by the expression of positive and/or negative molecular markers. The MSCs or the AD-MSCs may be identifiable at any passage discussed above. Positive molecular markers may be any one or more of the group including CD73, CD90 and CD105. Negative molecular markers may be any one or more of the group including CD34, CD116, CD19 and CD45. The stem cells may be derived from the subject so as to promote compatibility and/or inhibit rejection of the stem cells by the subject. Therefore, the stem cells may be autologous. Alternatively, the stem cells may be allogeneic.

The medium may be in the form of a growth medium or culture medium. The medium may be a settable medium. The medium, in an unset form, may have a viscosity which allows infiltration of at least a portion of the support arrangement. It is to be appreciated that when the medium is introduced into the support arrangement, the medium, typically being in an unset form, has a viscosity which facilitates absorption thereof by the support arrangement due to its porous characteristics, preferably through capillary action. Further, the medium may be introduced into the support arrangement in a sufficient quantity which allows the medium to surround the support arrangement. The medium may be protein-free. The medium may be in the form of any one or more of the group including, a neural basal medium, a neural induction medium, saline medium, phosphate-buffered saline medium, wash medium, holding medium, stem cell based medium, Roswell Park Memorial Institute Medium, Dulbecco's Modified Eagle's Medium, Dulbecco's Modified Eagle's Medium/F12, HAM's/F12 medium and HAM's/F10 medium. The medium may include additives. The additive may be in the form of any one or more of the group including, essential amino acids, vitamins and minerals. A second or subsequent medium may be provided, wherein each of the mediums are arranged in layers within or around the support arrangement. Further, each of the mediums may be different in terms of its form and/or the additives included therein.

The growth factor, preferably a plurality thereof, when in contact with the stem cell, may encourage and control development of the stem cell in the implant and/or any other endogenous stem cell in a region of the implant zone. The growth factor may have a reduced or no effect on the fully differentiated stem cells of the implant, more specifically, the fully differentiated oligodendrocytes or Schwann cells. Endogenous stem cells may be in the form of neural progenitor cells. The growth factor when in contact with the stem cells and/or the endogenous stem cells may encourage and control differentiation into any one or more cells of the group including neurons, astrocytes, oligodendrocytes, interneurons, microglia and Schwann cells. The growth factors may be a constituent of the medium. The growth factors may be in the form of any one or more of the group including, enzymes with kinase or phosphatase activity, tyrosine kinase activity, cytokines, lipid-soluble steroid hormones, nitric oxide, oligodendrocyte growth factor and reactive oxygen species (ROS). The growth factor may be in the form of a brain-derived neurotrophic factor (BDNF), neurotrophin 3, neurotrophin 4 and/or glial-cell-line derived neurotrophic factor (GDNF). The brain-derived neurotrophic factor may have a concentration in the range of 0.2 to 0.8 μl per 50 ml of medium, preferably being 0.5 μl per 50 ml of medium. The neurotrophin 3 or 4 may have a concentration in the range of 5 to 15 μl per 50ml of medium, preferably being 10 μl per 50 ml of medium and the GDNF may have a concentration in the range of 5 to 15 μg/ml, preferably being 10 μg/ml. The second or subsequent medium may include different growth factors for encouraging and controlling development of the stem cells and/or endogenous stem cells differently.

A cover may be provided for covering at least a portion of the support arrangement having received the stem cells, the medium and the growth factors. The cover may facilitate retarded resorption of the support arrangement post-implantation. The cover may hinder dislodgement of any one or more of the stem cells, the medium and the growth factors post-implantation, wherein the dislodgment may be caused by the flow of the subject's bodily fluids. The cover may facilitate guidance of the subject's bodily fluids away from the support arrangement. The bodily fluid may be in the form of the subject's cerebrospinal fluid or blood. The cover may be synthesised or manufactured from a porous and/or fibrous material for allowing at least some absorption of the subject's bodily fluids and in turn seepage of the bodily fluids toward the support arrangement. It is to be appreciated that seepage of the bodily fluids may provide added nutrients to the stem cells of the implant as well as movement of endogenous stem cells in a region of the implant. The cover may be synthesised or manufactured from a material of the kind as hereinbefore described. The cover may be in the form of a sheet, more particularly, a rectangular sheet. The sheet may be sized to have a length in the range of 6 to 10 mm, preferably being 7.5 mm. The sheet may be sized to have a thickness in the range of 1 to 2 mm, preferably being 1 mm. The sheet may be sized to have a width in the range of 2 to 4 mm, preferably being 2.5 mm. The cover may be in the form of an RCT Resorbable Collagen Tape®.

Alternatively, the cover may be in the form of a coating medium which coats at least a portion of the support arrangement having received the stem cells, the medium and the growth factors. The coating medium may be a settable medium. The coating medium in a set form, may be stiffer than the medium received by the support arrangement. The coating medium may be permeable for allowing permeation of the subject's bodily fluids toward the support arrangement.

According to a second aspect of the invention, there is provided an implant including:
a support arrangement;
receiving zones defined in the support arrangement for receiving stem cells in a supporting condition; and
a medium included in at least a portion of the support arrangement having received the stem cells in the supporting condition.

The support arrangement may receive one or more growth factors for controlling development of the stem cells.

The support arrangement may be capable of being sized and shaped to conform to and be received complementally by an implant zone in a subject.

The support arrangement, the receiving zones, the stem cells, the medium, the growth factors and the implant zone may be of the kind as hereinbefore described.

A cover may be provided for covering at least a portion of the support arrangement having received the stem cells, the medium and the growth factors. The cover may be of the kind as hereinbefore described.

The subject may be a mammal.

According to a third aspect of the invention, there is provided an implant including:
a support arrangement for supporting partially differentiated stem cells in a supported condition.

The support arrangement may receive a medium which includes nutrients and one or more growth factors for encouraging and controlling development of the stem cells.

The support arrangement may be capable of being sized and shaped to conform to and be received complementally by an implant zone in a subject.

The support arrangement, the stem cells, the medium, the growth factors and the implant zone may be of the kind as hereinbefore described.

A cover may be provided for covering at least a portion of the support arrangement having received the stem cells, the medium and the growth factors. The cover may be of the kind as hereinbefore described.

The subject may be a mammal.

According to a fourth aspect of the invention, there is provided an implant as hereinbefore described, for use in regenerative medical treatments.

The regenerative medical treatments may include any one or more of the group including spinal cord regenerative treatments, nerve regenerative treatments, cartilage regenerative treatments, bone regenerative treatments, soft tissue regenerative treatments, regenerative treatment of Alzheimer's, regenerative treatment of dementia, regenerative treatment of diabetes, treatment of macular degeneration, treatment of glaucoma, nephron regenerative treatments, regenerative treatment of multiple sclerosis, treatment of cancer and any autoimmune disease.

The regenerative medical treatments may be suitable for mammals.

According to a fifth aspect of the invention, there is provided a method for the regenerative treatment of injury or disease, the method includes:
implanting an implant, as hereinbefore described, in an implant zone defined in a subject.

The implant zone may be a site of injury, damage or deterioration of tissue in the subject's body. The implant zone may be a space defined by any one of the group including a damaged, deteriorated, severed, torn and excised tissue in the subject's body. More specifically, the implant zone may be a space defined by the excision of a portion of the subject's spinal cord. Alternatively, the implant zone may be a space defined by a severed, torn, broken or excised portion of the subject's peripheral nerve. The subject may be a mammal.

According to a sixth aspect of the invention, there is provided a method for preparing an implant, the method including:
obtaining stem cells;
introducing the stem cells into a support arrangement, wherein the stem cells are supported by the support arrangement in a supported condition;
introducing a medium into the support arrangement, wherein the medium provides nourishment to the stem cells in the supported condition; and
introducing a growth factor into the support arrangement, wherein the growth factor controls and encourages development of the stem cells in the supported condition.

The stem cells may be obtained from tissue obtained from a subject in need of the implant. The stem cells may be autologous or allogeneic. The stem cells may be obtained from the tissue using any known technique. The stem cells may be of the kind as hereinbefore described. To determine if stem cells were obtained or to identify a type or form of stem cells obtained, the method may include conducting quantitative polymerase chain reaction (qPCR) tests to ascertain if the stem cells or potential stem cells are expressing molecular marks indicative of being a stem cell or a stem cell of interest. To identify the presence of MSCs or more specifically AD-MSCs, qPCR is conducted to detect positive and/or negative molecular markers. The positive molecular markers may be any one or more of the group including, CD73, CD90 and CD105. The negative molecular markers may be any one or more of the group including CD34, CD116, CD19 and CD45.

The method may include fully differentiating the stem cells into a cell-type or tissue type of interest using any known technique. The cell-type of interest may be in the form of oligodendrocytes or Schwann cells. More specifically, the oligodendrocytes may be in the form of oligodendrocytes progenitor cells (OPCs) and/or premyelinating oligodendrocytes. Fully differentiating the stem cells into oligodendrocytes may include the following steps:
1) plating undifferentiated stem cells in a culture plate using culture medium;
2) incubating the plated stem cells for a period of 2 to 5 days, typically being 3 days, at a temperature in the range of 37 to 37.5° C.;
3) priming the stem cells using a priming medium;
4) initiating stem cell differentiation using a differentiation medium;
5) fully differentiating the stem cells using a final differentiation medium; and
6) determining whether the stem cells are differentiating or have differentiated into oligodendrocytes.

In step (1) above, the culture medium may be in the form of DMEM/F12. The DMEM/F12 may include FBS and/or Gentamycin. The FBS and Gentamycin may have concentrations in the range of 10 to 15% and 1 to 2% respectively. Preferably, the FBS and Gentamycin may have concentrations of 10% and 1% respectively.

In step (3) above, the priming medium may be in the form of any suitable priming medium. The priming medium may include Trypsin and Ethylenediaminetetraacetic acid (EDTA) at a concentration in the range of 0.2 to 0.3% [v/v] and 0.01 to 0.03% [v/v], respectively. Preferably, the concentration of Trypsin and EDTA may be 0.25% [v/v] and 0.02% [v/v], respectively.

In step (4) above, the differentiation medium may be in the form of any suitable medium. The differentiation medium may include Retinoic acid and/or Sonic hedgehog protein (SHH). The Retinoic acid may be added into the differentiation medium from a stock solution having a concentration in the range of 18 to 22 mM, preferably being 20 mM. The Retinoic acid stock solution may be added to the differentiation medium in a ratio of 0.01:0.1 ml. The SHH may be added to the differentiation medium from a stock solution having a concentration in the range of 23 μg/L to 27 μg/L, preferably being 25 μg/L. The SHH stock solution may be added to the differentiation medium in a ratio of 1:100 ml.

In step (5) above, the final differentiation medium may be in the form of any suitable final differentiation medium. The final differentiation medium may include neurotrophin 3 and/or platelet-derived growth factor (PDGF). The PDGF may be added into the final differentiation medium from a stock solution having a concentration of 1 μg/mL. The PDGF stock solution may be added to the final differentiation medium in a ratio of 1:50 ml. The PDGF may be in the form of PDGFα. The neurotrophin 3 may be added into the final differentiation medium from a stock solution having a concentration of 1 μg/mL. The neurotrophin 3 stock solution may be added to the final differentiation medium in a ratio of 1:33 ml.

In step (6) above, qPCR testing may be employed to determine whether the stem cells are expressing molecular markers which are characteristic of oligodendrocytes. The molecular markers may include any one or more of the group including SOX 10, OLIG1, OLIG2, NKX2.2, ZFP191, ZFP488, ZFP536, SOX17 and NKX 6.2.

The support arrangement may be of the kind as hereinbefore described. The support arrangement may be prepared by puncturing it to form one or more holes for allowing insertion of the stem cells into an inner region of the support arrangement. It is to be appreciated that the hole serves to encourage filling, saturation and/or distribution of the stem cells throughout the support arrangement. The holes may extend substantially co-axially the support arrangement. The holes may extend varying depths within the support arrangement, preferably two thirds a length of the support arrangement. The support arrangement may be punctured using a thin elongate member such as a needle.

The stem cells may be introduced into the support arrangement using a stem cell suspension. The stem cell suspension may include a mixture of stem cells at various stages of differentiation. The mixture may include any one or more of undifferentiated, determined, partially differentiated and fully differentiated stem cells. Preferably, the mixture may include undifferentiated and fully differentiated stem cells. The fully differentiated stem cells may be in the form of oligodendrocytes or Schwann cells. The oligodendrocytes may be in the form of oligodendrocytes progenitor cells (OPCs) and/or premyelinating oligodendrocytes. The stem cell suspension may be introduced into the support arrangement via the hole or holes in a drip-like manner or by pouring.

The medium may be of the kind as hereinbefore described. The medium may be introduced into the support arrangement by submerging at least a portion of the support arrangement into unset medium. Preferably, the medium may be introduced into the support arrangement by submerging it entirely into the unset medium. Alternatively, the medium, in an unset form, may be introduced into the support arrangement, preferably via the hole, in a drip-like manner or by pouring. Thereafter, the medium may be allowed to set. In a set form, the medium may allow for the stem cells to be held in position relative the support arrangement in the supported condition.

The growth factor, preferably a plurality thereof, may be of the kind as hereinbefore described. The growth factors may be introduced into the support arrangement, preferably via the hole or holes, in a drip-like manner or by pouring. Preferably, the growth factors may be a constituent of the medium.

The method may include allowing the undifferentiated, determined and/or partially differentiated stem cells to develop in the supported condition before implantation into the subject. The stem cells may be allowed to develop until any stage during its differentiation toward being a specific cell-type of interest. The stage of differentiation may be any one or more of the group including, undifferentiated, determined, partially differentiated and fully differentiated stem cells, preferably being partially differentiated. The partially differentiated stem cell may exhibit early morphologically distinct characteristics of neural cells. The characteristic may be the formation of an axon hillock. The partially differentiated stem cells may express molecular markers indicative of a tissue-type or cell-type of interest.

The method may include monitoring the development of the stem cells which are allowed to develop in the supported condition before implantation into the subject. The stem cells may be monitored to identify a desired stage of differentiation toward being a specific cell-type of interest. The stage of differentiation may be any one or more of the group including, undifferentiated, determined, partially differentiated and fully differentiated stem cells. Preferably, the stage of differentiation may be partially differentiated stem cells. Monitoring may include culturing a portion of the stem cells separately to the stem cells included in the implant. Culturing the stem cells, to be monitored, may be initiated at the same time as the stem cells introduced into the implant, using a medium and a growth factor as used in the implant. Further, monitoring may include periodical viewing of the cultured stem cells using light microscopy to identify early distinctive characteristics of the desired cell-type and/or conducting qPCR tests to ascertain if the stem cells are expressing molecular marks which indicate differentiation toward the desired cell-type. The early distinctive characteristic may be an axon hillock. The molecular markers may include any one or more of the group including NeuN, Tuji, Synapsin, PAX6, SOX1, SOX2, bActin, NESTIN, MAP2 and/or GFAP.

The method includes wrapping or covering at least a portion of the support arrangement having received the stem cells and/or the medium and/or the growth factors with a cover of the kind as hereinbefore described.

### BRIEF DESCRIPTION OF THE DRAWINGS

An implant in accordance with the invention will now be described by way of the following, non-limiting examples with reference to the accompanying drawings.

In the drawings:

FIG. 1 is a cross-sectional view of a first embodiment of an implant in accordance with the invention;

FIG. 2 is a three-dimensional schematic showing the implant of FIG. 1;

FIG. 3 is a three-dimensional schematic showing a support arrangement in the form of a porous material, wherein an insert of a scanning electron micrograph of the material is provided;

FIG. 4 is a three-dimensional schematic showing part of a spinal cord having an excised portion;

FIGS. 5 and 6 are three-dimensional schematics sharing different arrangements of the implant in an implant zone;

FIGS. 7 and 9 is a three-dimensional schematic showing stem cell development within the implant when in use;

FIG. 8 is a three-dimensional schematic showing the implant of FIG. 1 with a cover in accordance with the invention; and

FIG. 10 is a schematic showing a partially differentiated stem cell including an axon hillock.

### DETAILED DESCRIPTION OF THE INVENTION

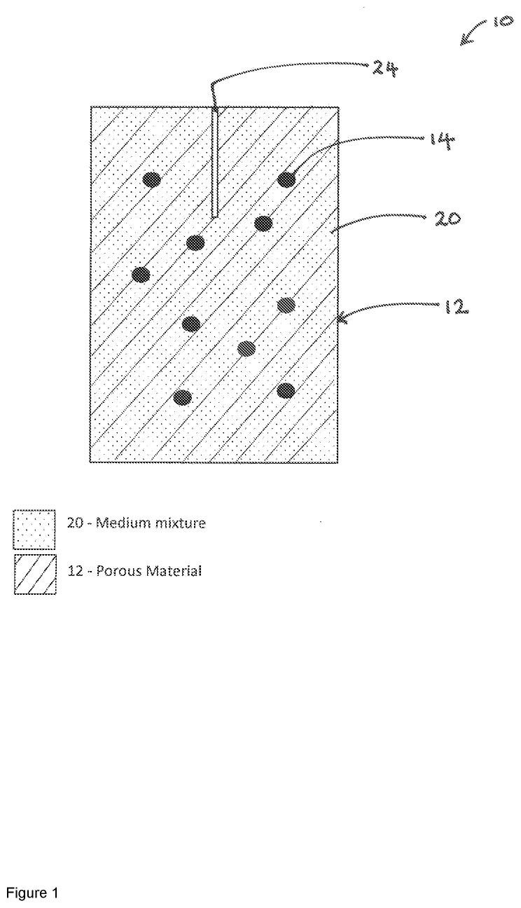

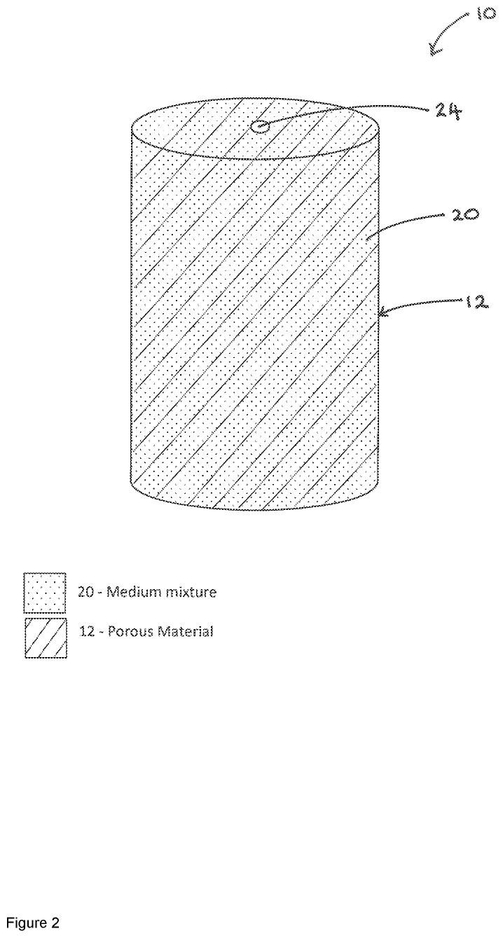

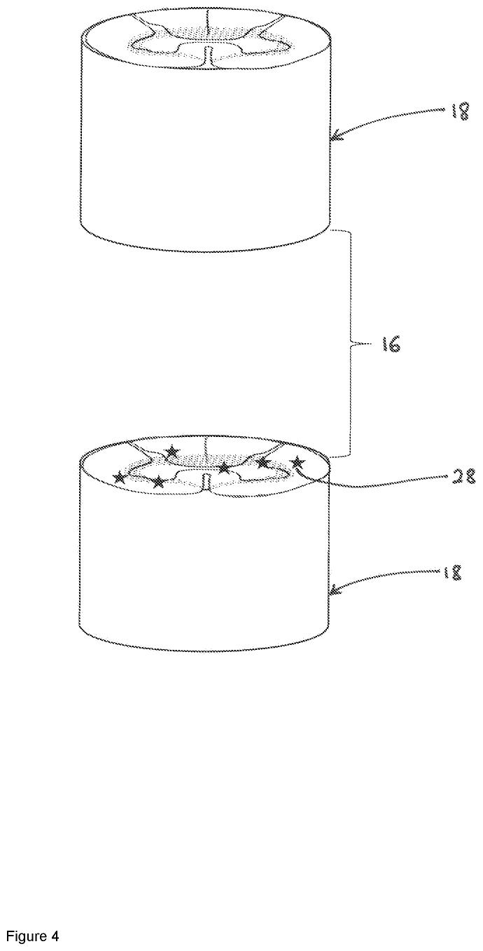

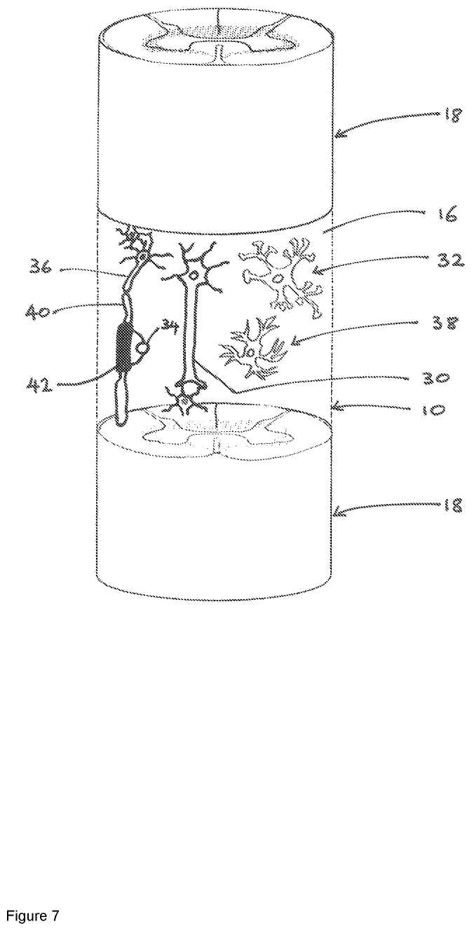

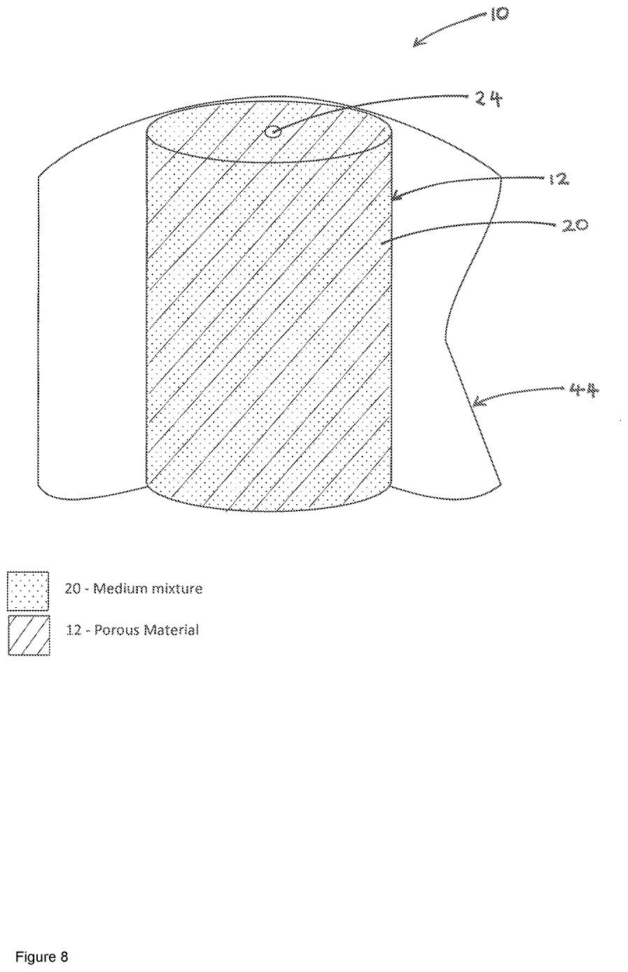

In a first embodiment of the invention, shown in FIGS. 1 to 8, reference numeral 10 refers generally to an implant.

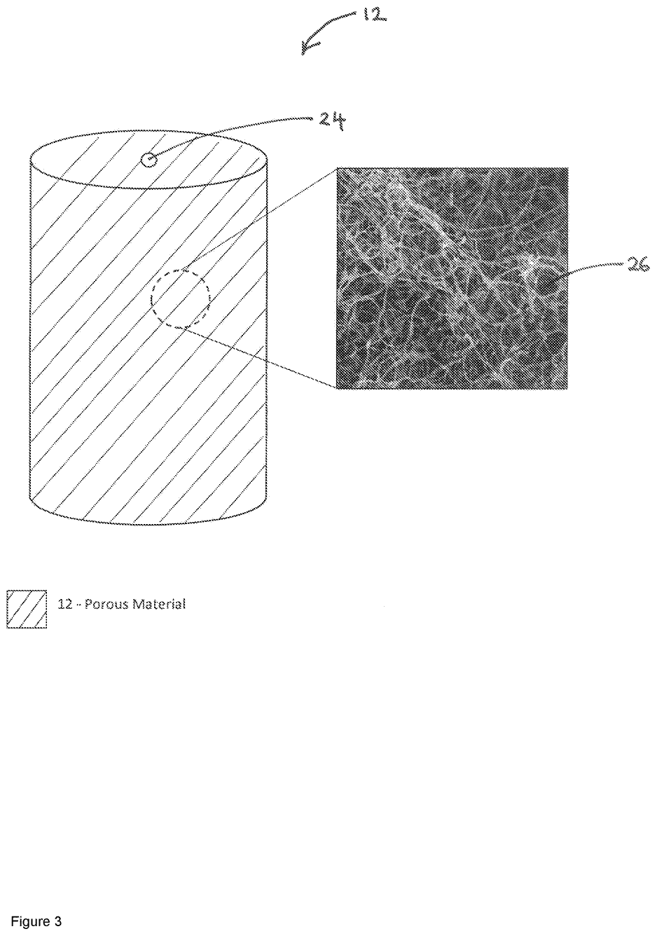

In this embodiment, the implant 10 includes a support arrangement 12 manufactured from a form of a porous material, as clearly shown in FIG. 3, for receiving and supporting stem cells 14 in a supported condition, for receiving a medium in the form of a neural basal and neural induction medium mixture 20, the support arrangement 12 being shaped and sized to conform to and be received complementally by an implant zone 16, the implant zone 16 being a space defined by an excised portion of a subject's spinal cord 18 as shown in the Figures. The neural basal and neural induction medium mixture 20 provides nutrients for the development of the stem cells 14 and growth factors in form of neurotrophin 3, neurotrophin 4 and brain-derived neurotrophic factor (BDNF) for encouraging and controlling development of the stem cells 14, the growth factors being constituents of the medium mixture 20.

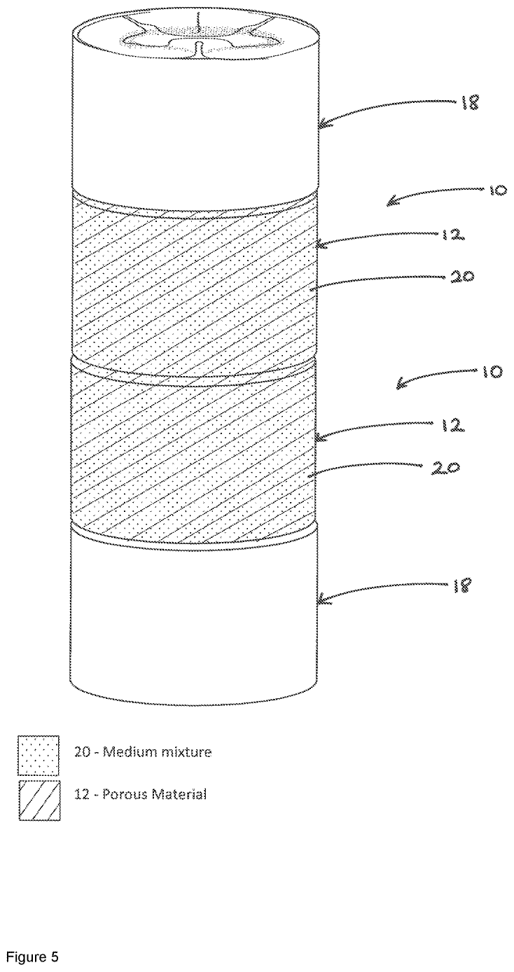

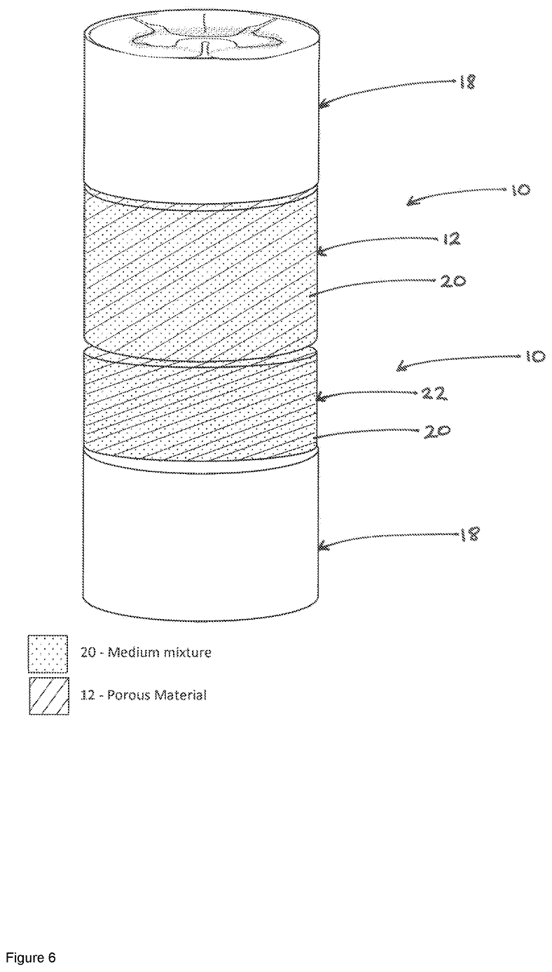

The support arrangement 12 is a natural polymer which is in the form of collagen. More specifically, the support arrangement is in the form of resorbable collagen, even more specifically, being in the form of resorbable purified crosslinked type 1 collagen. Typically, the resorbable purified crosslinked type 1 collagen is resorbed by the subject within approximately 30 days post-implantation. The resorption of the collagen allows for unhindered continued development of the stem cells 14 whilst providing support for the developing stem cells in its early fragile stages of development. The collagen is derived from bovine Achilles tendon and is in the form of an RCP Resorbable Collagen PLUG®. The RCP Resorbable Collagen PLUG® has dimensions of 1 cm×2 cm. When a length of the implant zone 16 exceeds the length of the support arrangement 12, multiple support arrangements 12 may be provided in an end-to-end arrangement, as clearly shown in FIG. 5. Alternatively, a portion of a second support arrangement 22 may be cut to a desired length and arranged end-to-end with the first support arrangement 12, as clearly shown in FIG. 6. A hole 24 is defined in the support arrangement 12 for allowing insertion of the stem cells 14 into an inner region thereof. It is to be appreciated that the hole 24 serves to encourage filling, saturation and distribution of the stem cells 14 substantially throughout the support arrangement 12. The hole 24 is formed by the use of a thin elongate member such as a needle. Although not shown in the Figures, a plurality of holes can be defined in the support arrangement 12 for further encouraging filling, saturation and distribution of the stem cells substantially throughout the support arrangement 12. The plurality of holes may be of differing depths of up to two thirds the length of the support arrangement 12.

The support arrangement 12 defines receiving zones in the form of pores 26, most clearly shown in FIG. 3. The stem cells 14 are received within the pores 26 and in turn, are supported by the support arrangement 12. The pores 26 have a general width of 300 to 600 μm. The support arrangement 12 is hygroscopic and adsorbent and will absorb moisture when exposed to the atmosphere and/or the medium mixture 20. Upon hydration and/or absorption, the pores 26 have a general width of 150 to 300 μm. A reduction in size of the pores 26, upon hydration and/or absorption, facilitates support of the stem cells 14 in the supported condition.

The stem cells 14 are in the form of multipotent stem cells, specifically mesenchymal stem cells (MSCs), and more specifically, adipose-derived mesenchymal stem cells (AD-MSCs). The MSCs or the AD-MSCs are identifiable by the expression of positive and negative molecular markers. The positive molecular markers are CD73, CD90 and CD105. The negative molecular markers are CD34, CD116, CD19 and CD45. The stem cells 14 are a mixture of primarily partially differentiated and fully differentiated stem cells 14. The partially differentiated stem cells 14 exhibit morphologically distinct characteristics of neural cells. The characteristic is the formation of an axon hillock 50. The partially differentiated stem cells express one or more molecular markers specific to neural cells. The molecular markers are one or more of the group including NeuN, Tuji, Synapsin, PAX6, SOX1, SOX2, bActin, NESTIN, MAP2 and/or GFAP. The fully differentiated stem cells 14 are in the form of oligodendrocytes 34, more specifically, oligodendrocytes progenitor cells (OPCs) and premyelinating oligodendrocytes. The oligodendrocytes 34 express one or more molecular markers of the group including SOX 10, OLIG1, OLIG2, NKX2.2, ZFP191, ZFP488, ZFP536, SOX17 and NKX 6.2. Including fully differentiated stem cells 14 in the mixture, ensures a presence of a specific type of cell in a sufficient quantity in the implant 10 and in turn, the implant zone 16, post-implantation. A concentration of the stem cells 14 is 2.5 million per 1 cm³ of support arrangement 12 and are therefore provided for in surplus to allow for differentiation of the stem cells 14 into a cell-type determined by the growth factors or any other cell-type determined by cell-to-cell signalling or endogenous signalling post-implantation. The fully differentiated stem cells 14 comprise approximately 3% of a total number of stem cells 14. The stem cells 14 are obtained during any of passages 3 to 9. More specifically, the fully differentiated stem cells 14 are obtained during passage 5 or 6.

The medium mixture 20 is a settable medium. The medium mixture 20 in an unset form, has a viscosity which allows for infiltration of at least a portion of the support arrangement 12. It is to be appreciated that when the unset medium mixture 20 is introduced into the support arrangement 12, it has a viscosity which facilitates absorption thereof by the support arrangement 12 due to its porous characteristics, typically through capillary action. Further, the medium mixture 20 is introduced into the support arrangement 12 in a sufficient quantity which allows for the medium mixture 20 to surround the support arrangement 12. The medium mixture 20 when received by the support arrangement 12 and in a set form, facilitates support of the stem cells 14 in a position relative to the support arrangement 12 in the supported condition.

The growth factors when in contact with the stem cells 14 encourage and control development of the stem cells 14 in the implant 10 and can have little or no effect on the fully differentiated stem cells 14 and more specifically the fully differentiated oligodendrocytes 34. The growth factors when in contact with endogenous stem cells 28 in a region of the implant zone 16, encourage and control development of the endogenous stem cells 28. The endogenous stem cells 28 typically being in the form of neural progenitor cells which are released from the central canal during excision of the spinal cord 18. Development of the stem cells 14 and/or the endogenous stem cells 28 includes any one or more of the group including differentiation, continued differentiation and proliferation and may include cell migration or multicellular morphogenesis. The stem cells 14 and/or the endogenous stem cells 28 when differentiated are any one or more of the group including neurons 30, astrocytes 32, oligodendrocytes 34, interneurons 36 and microglia 38. The inclusion of partially differentiated stem cells 14 in the implant 10, facilitates survival and successful differentiation post-implantation as well as hindered neurotoxin release which occurs during synapsis which occurs in fully developed neurons 30 when being handled. The medium mixture 20 together with the growth factors may encourage regeneration of the subject's axons 40. The implant 10 may provide conditions suitable for myelination 42 of any one or more of differentiating or differentiated stem cells 14 into neurons 30, the differentiated or differentiated endogenous stem cells 28 into neurons 30 and the regenerated axons 40. The oligodendrocytes 34 may facilitate myelination 42 of the regenerated axons 40 and/or differentiated neurons 30 as well as the remyelination of endogenous neurons 40 which may be or have been damaged.

The implant 10 includes a cover in the form of a rectangular sheet 44 for covering a portion of the support arrangement 12 having received the stem cells 14, the medium mixture 20 and the growth factors. The sheet 44 facilitates retarded resorption of the support arrangement 12 post-implantation which increases the time of support provided by the support arrangement, as well as hindered dislodgement of any one or more of the stem cells 14, the medium mixture 20 and the growth factors post-implantation, wherein the dislodgment can be caused by the flow of the subject's cerebrospinal fluid. The sheet 44 is in the form of a porous material for allowing absorption of the subject's cerebrospinal fluid and in turn seepage toward the support arrangement 12. The sheet 44 is sized to have a length of 7.5 mm, a thickness of 1 mm and a width of 2.5 mm. The sheet 44 is in the form of an RCT Resorbable Collagen Tape®.

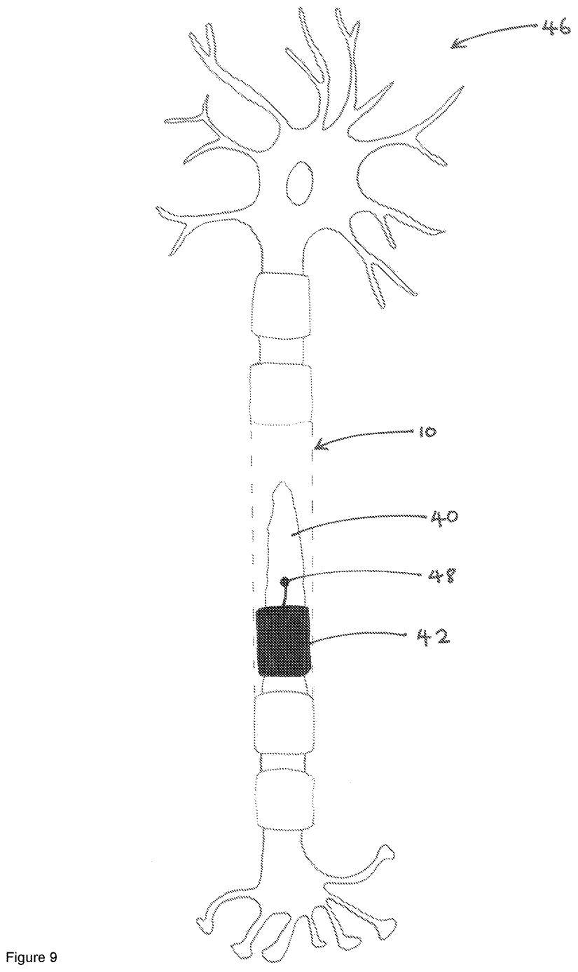

In a second embodiment of the invention, shown in FIG. 9, the implant 10 may conform to an implant zone 16 in the form of a space defined by a severed peripheral nerve 46. The growth factors control and encourage regeneration of an axon 40 of the peripheral nerve 46. The stem cells 14 are a mixture of primarily partially differentiated and fully differentiated stem cells 14, wherein the fully differentiated stem cells 14 are Schwann cells 48. Further, the growth factors control and encourage continued differentiation of the partially differentiated stem cells 14 into Schwann cells 48. The Schwann cell 48 can facilitate myelination 42 of the regenerating axon 40.

A third embodiment of the invention relates to a method for preparing the implant 10, the method including:
obtaining stem cells 14;
introducing the stem cells 14 into a support arrangement 12 manufactured from a porous material, wherein the stem cells 14 are supported by the support arrangement 12 in a supported condition;
introducing a medium in the form of a neural basal and neural induction medium mixture 20 into the support arrangement 12, wherein the medium mixture 20 provides nourishment to the stem cells 14 in the supported condition; and
introducing a growth factor in the form of neurotrophin 3, neurotrophin 4 and BDNF into the support arrangement 12, wherein the growth factor controls and encourages development of the stem cells 14 in the supported condition.

The stem cells 14 are obtained from tissue obtained from a subject in need of the implant. Therefore, the stem cells 14 are autologous. The stem cells 14 are obtained using known techniques. The stem cells 14 are MSCs and more specifically, AD-MSCs. To determine if the stem cells 14 are MSCs or more specifically AD-MSCs, quantitative polymerase chain reaction (qPCR) tests are conducted to detect an expression of positive and negative molecular markers specific to MSCs or AD-MSCs. The positive molecular markers are CD73, CD90 and CD105. The negative molecular markers are CD34, CD116, CD19 and CD45.

The method includes fully differentiating some stem cells 14 into a cell-type or tissue type of interest. The cell-type of interest is in the form of oligodendrocytes 34, more specifically, oligodendrocytes progenitor cells (OPCs) and premyelinating oligodendrocytes. Fully differentiating the stem cells 14 into oligodendrocytes 34 includes the following steps:
1) plating undifferentiated stem cells 14 in a culture plate using culture medium;
2) incubating the plated stem cells 14 for a period of 3 days, at a temperature of 37° C.;
3) priming the stem cells 14 using a priming medium;
4) initiating stem cell 14 differentiation using a differentiation medium;
5) fully differentiating the stem cells 14 using a final differentiation medium; and
6) determining whether the stem cells 14 are differentiating or differentiated into oligodendrocytes 34.

In step (1) above, the culture medium is in the form of DMEM/F12, including FBS and/or Gentamycin. The FBS and Gentamycin have concentrations of 10% and 1% respectively.

In step (3) above, the priming medium is in the form of any suitable priming medium. The priming medium includes Trypsin and Ethylenediaminetetraacetic acid (EDTA) at a concentration of 0.25% [v/v] and 0.02% [v/v] respectively. The priming medium is at a temperature in the range of 37 to 37.5° C., preferably being 37° C. The stem cells 14 are added to the priming medium to form a cell suspension. The cell suspension is incubated for a period in the range of 3 to 6 mins, preferably being 5 mins. Thereafter, the cell suspension is centrifuged to form a pellet of stem cells 14. The pellet is resuspended in fresh priming medium. The resuspended stem cells 14 are plated in culture plates and incubated at a temperature in the range of 37 to 37.5° C., preferably being 37° C. The stem cell 14 are incubated for a period of 18 days. During the incubation period the priming medium is at least partially removed and replaced with fresh priming medium periodically, preferably being every 7 days.

In step (4) above, the differentiation medium is in the form of any suitable differentiation medium. The differentiation medium comprises Retinoic acid and/or Sonic hedgehog protein (SHH). The Retinoic acid is added into the differentiation medium from a stock solution having a concentration of 20 mM. The Retinoic acid stock solution is added to the differentiation medium in a ratio of 0.01:0.1 ml. The SHH is added to the differentiation medium from a stock solution having a concentration of 25 μg/L. The SHH stock solution is added to the differentiation medium in a ratio of 1:100 ml. The stem cells 14 are plated in culture plates using the differentiation medium. The plated stem cells 14 are incubated for a period of 10 days at a temperature in the range of 37 to 37.5° C., preferably being 37° C. During the incubation period the differentiation medium is at least partially removed and replaced with fresh differentiation medium periodically, preferably being every 7 days.

In step (5) above, the final differentiation is in the form of any suitable final differentiation medium. The final differentiation medium comprises neurotrophin 3 and platelet-derived growth factor (PDGF). The PDGF is in the form of PDGFα. The PDGFα is added into the final differentiation medium from a stock solution having a concentration of 1 μg/mL. The PDGFα stock solution is added to the final differentiation medium in a ratio of 1:50 ml. The neurotrophin 3 is added into the final differentiation medium from a stock solution having a concentration of 1 μg/mL. The neurotrophin 3 stock solution is added to the final differentiation medium in a ratio of 1:33 ml. The stem cells 14 are plated in culture plates using the final differentiation medium. The plated stem cells 14 are incubated for a period of 14 days at a temperature in the range of 37 to 37.5° C., preferably being 37° C. During the incubation period the final differentiation medium is at least partially removed and replaced with fresh final differentiation medium periodically, preferably being every 7 days.

In step (6) above, qPCR testing is employed to determine whether the stem cells 14 are expressing molecular markers which are characteristic of oligodendrocytes 34. The molecular markers include any one or more of SOX 10, OLIG 1, OLIG 2, NKX 2.2, ZFP 191, ZFP 488, ZFP 536, SOX 17 and NKX 6.2. More specifically, the molecular markers include any one or more of SOX 10, NKX 2.2, OLIG1 and OLIG2 for OPCs 34 and OLIG1, OLIG2, SOX10, NKX2.2, ZFP191, ZFP488, ZFP536, SOX17 and NKX 6.2 for premyelinating oligodendrocytes 34.

The method includes puncturing the support arrangement 12 using a hypodermic needle (not shown) to form a hole 24 to allow insertion of the stem cells 14 into an inner region of the support arrangement 12. The hole 24 is defined substantially co-axially the support arrangement 12.

The stem cells 14 are introduced into the support arrangement 12 using a stem cell suspension (not shown). The stem cell suspension is introduced into the support arrangement 12 in a drip-like manner via the hole 24.

The medium mixture 20 is introduced into the support arrangement 12 by submerging the support arrangement 12 into the medium mixture 20 when in an unset form. Thereafter, the medium mixture 20 is allowed to set thereby supporting the stem cells 14 in a position relative the support arrangement 12 in the supported condition.

The growth factors are introduced into the support arrangement 12 together with the medium mixture 20. The growth factors are constituents of the medium mixture 20.

The method includes allowing the stem cells 14 which were not fully differentiated, to develop in the set medium mixture 20 in the supported condition. These stem cells could be undifferentiated and/or determined cells. The stem cells 14 are allowed to develop until partially differentiated. The stem cells 14 are partially differentiated to be any one or more of the group including neurons 30, astrocytes 32, oligodendrocytes 34, interneurons 36 and Schwann cells 48. The inclusion of partially differentiated stem cells 14 in the implant 10, facilitates successful survival and development post-implantation as well as hindered neurotoxin release during synapsis which occurs in fully developed neurons 30 when being handled.

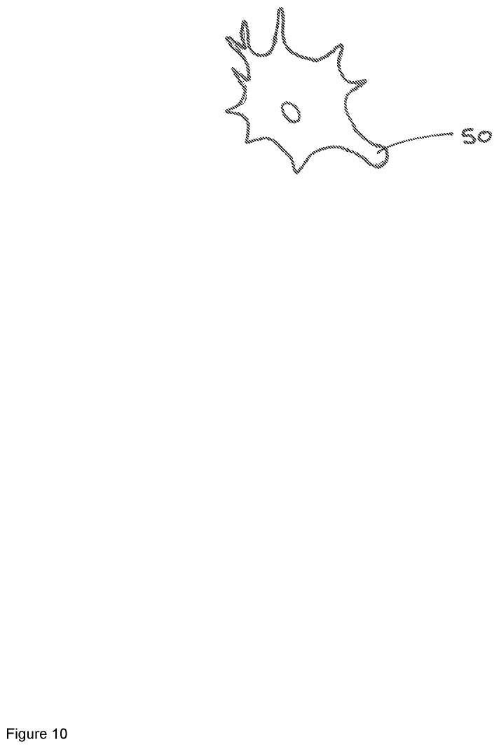

The method includes monitoring the development of the stem cells 14 which are allowed to develop until partially developed. Monitoring includes culturing a portion of the stem cells 14 separately to the stem cells 14 included in the implant 10. Culturing of stem cells 14 to be monitored is initiated at the same time as when the stem cells 14 are introduced into the implant 10. Monitoring involves periodical viewing of the cultured stem cells 14 using light microscopy to identify early distinctive characteristics of a desired cell-type and/or conducting qPCR tests to ascertain if the stem cells 14 are expressing molecular markers which indicate differentiation toward a desired cell-type which are neurons. The early distinctive characteristics is the formation of an axon hillock 50 shown in FIG. 10. The molecular markers include any one or more of the group including NESTIN, PAX6, SOX1, SOX2, bActin, NeuN, TujI, Synapsin, MAP2 and/or GFAP.

Although only certain embodiments of the invention have been described herein, it will be understood by any person skilled in the art that other modifications, variations, and possibilities of the invention are possible. Such modifications, variations and possibilities are therefore to be considered as falling within the spirit and scope of the invention and hence form part of the invention as herein described and/or exemplified. It is further to be understood that the examples are provided for illustrating the invention further and to assist a person skilled in the art with understanding the invention and is not meant to be construed as unduly limiting the reasonable scope of the invention.

The inventor believes that the implant in accordance with the present invention is advantageous in that the implant provides for the amelioration of injured, damaged and/or deteriorating tissue by regeneration. It allows for ease of handling of stem cells during implantation. The use of partially differentiated stem cells facilitates survival and successful development of stem cells post-implantation as well as hindered neurotoxin release during synapsis of fully developed neurons which often occurs during handling. Further, the implant provides support for developing stem cells which in turn facilitates integration into the environment in which is it implanted.

1. An implant comprising:
a support arrangement for,
receiving and supporting stem cells in a supporting condition;
receiving a medium which includes nutrients; and
receiving a growth factor for encouraging and controlling development of the stem cells,
wherein the support arrangement is capable of being sized and shaped to conform to and be received complementally by an implant zone in a subject.

2. (canceled)

3. (canceled)

4. (canceled)

5. An implant as claimed in claim 1, wherein the support arrangement has an internal lattice-type structure.

6. (canceled)

7. (canceled)

8. (canceled)

9. (canceled)

10. An implant as claimed in claim 1, wherein a hole is defined in the support arrangement for allowing insertion of the stem cells into an inner region thereof, wherein the hole extends substantially co-axially the support arrangement and typically two thirds a length of the support arrangement.

11. (canceled)

12. (canceled)

13. (canceled)

14. (canceled)

15. (canceled)

16. An implant as claimed in claim 1, wherein the support arrangement is manufactured from a natural polymer in the form of a collagen.

17. (canceled)

18. (canceled)

19. An implant as claimed in claim 16, wherein the collagen is in the form of resorbable collagen including a crosslinked type 1 collagen.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. An implant as claimed in in claim 1, wherein the stem cells are at any stage of differentiation toward being a specific cell-type of interest, wherein the stem cells include a mixture of stem cells at various stages of differentiation.

41. (canceled)

42. (canceled)

43. (canceled)

44. (canceled)

45. An implant as claimed in claim 40, wherein the mixture includes stem cells selected form the group consisting of undifferentiated, determined, partially differentiated, and fully differentiated stem cells, wherein the partially differentiated stem cells are stem cells exhibiting early morphologically distinct characteristics of being neural cells.

46. (canceled)

47. (canceled)

48. An implant as claimed in claim 45, wherein the morphologically distinct characteristic is a formation of an axon hillock.

49. An implant as claimed in claim 45, wherein the partially differentiated stem cells are identifiable by an expression of any one or more molecular markers specific to neural cells, wherein the molecular markers specific to neural cells are selected from the group consisting of NeuN, Tuji, Synapsin, PAX6, SOX1, SOX2, bActin, NESTIN, MAP2 and GFAP.

50. (canceled)

51. An implant as claimed in claim 45, wherein the fully differentiated stem cells are in the form of oligodendrocytes or Schwann cells, wherein the oligodendrocytes are in the form of oligodendrocytes progenitor cells (OPCs) and/or premyelinating oligodendrocytes, and wherein the OPCs are identifiable by an expression of any one or more molecular markers of the group including, SOX 10, NKX 2.2, OLIG1 and OLIG2.

52. (canceled)

53. (canceled)

54. (canceled)

55. An implant as claimed in claim 1, wherein the stem cells have a concentration in a range of 150 000 to 1 500 000 per 1 cm³ of material of the support arrangement.

56. An implant as claimed in claim 55, wherein a surplus number of stem cells are provided for allowing differentiation of the stem cells into a cell-type of interest and/or any other cell-type, wherein the surplus number of stem cells have a concentration in the range of 2 000 000 to 3 000 000 per 1 cm3 of material of the support arrangement.

57. (canceled)

58. (canceled)

59. (canceled)

60. (canceled)

61. An implant as claimed in claim 1, wherein the stem cells are in the form of any one or more of the group including embryonic stem cells (ESCs), mesenchymal stem cells (MSCs), hematopoietic stem cells (HSCs), neural stem cells (NSCs), induced pluripotent stem cells (iPSCs), dental pulp stem cells (DPSCs), olfactory ensheathing cells (OECs), cancer stem cells (CSCs) and human foetal brain neural stem cells (hNSCs).

62. An implant as claimed in claim 61, wherein the MSCs are in the form of any one selected from the group including, bone marrow mesenchymal stem cells (BM-MSCs), human umbilical cord-mesenchymal stem cells (HUC-MSCs) and adipose-derived mesenchymal stem cells (AD-MSCs).

63. An implant as claimed in claim 61, wherein the NSCs are in the form of neural progenitors, neutral crest cells and/or Schwann cell precursors.

64. An implant as claimed in claim 61, wherein the stem cells are in the form of MSCs, wherein the MSCs are in the form of AD-MSCs, and wherein the AD-MSCs are identifiable by an expression of positive molecular markers selected from any one or more of the group including, CD73, CD90 and CD105.

65. (canceled)

66. (canceled)

67. An implant as claimed in claim 61, wherein the stem cells are in the form of MSCs, wherein the MSCs are in the form of AD-MSCs, and wherein the AD-MSCs are identifiable by an expression of negative molecular markers selected from any one or more of the group including, CD34, CD116, CD19 and CD45.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. An implant as claimed in claim 1, wherein the growth factor is selected to encourage and control development of the stem cells in the implant and/or any other endogenous stem cells in the region of the implant zone, wherein the growth factor is in the form of any one or more selected from the group including, enzymes with kinase or phosphatase activity, tyrosine kinase activity, cytokines, lipid-soluble steroid hormones, nitric oxide, oligodendrocyte growth factor and reactive oxygen species (ROS).

83. (canceled)

84. (canceled)

85. (canceled)

86. (canceled)

87. (canceled)

88. (canceled)

89. (canceled)

90. (canceled)

91. (canceled)

92. (canceled)

93. An implant as claimed in claim 1, including a cover for covering at least a portion of the support arrangement having received the stem cells, the medium and one or more growth factors.

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. A method for preparing an implant of claim 1, the method comprising:
obtaining stem cells;
introducing the stem cells into a support arrangement, wherein the stem cells are supported by the support arrangement in a supporting condition;
introducing a medium into the support arrangement, wherein the medium provides nourishment to the stem cells in the supporting condition; and
introducing a growth factor into the support arrangement, wherein the growth factor controls and encourages development of the stem cells in the supporting condition.

119-157. (canceled)
